(19) United States
(12) Patent Application Publication
(10) Pub. No.: US 2024/0074651 A1
(43) Pub. Date: Mar. 7, 2024

(54) METHOD AND DEVICE FOR MEASURING THE VISUAL FIELD OF A PERSON

(71) Applicant: H & M MEDICAL SOLUTIONS GMBH, Berlin (DE)

(72) Inventors: Friedrich HOFFMANN, Berlin (DE); Fabian MÜLLER, Berlin (DE)

(73) Assignee: H & M MEDICAL SOLUTIONS GMBH, Berlin (DE)

(21) Appl. No.: 18/261,275

(22) PCT Filed: Jan. 14, 2022
(86) PCT No.: PCT/EP2022/050765

§ 371 Date: Jul. 13, 2023

(30) Foreign Application Priority Data
Jan. 14, 2021 (EP) 21151704.0
May 27, 2021 (EP) 21176171.3
Sep. 13, 2021 (EP) 21196409.3

(51) Int. Cl. A61B 3/024

(52) U.S. Cl. CPC A61B 3/024; A61B 3/0041

(57) ABSTRACT

The invention relates to a method for measuring the visual field of a person by means of a device, comprising the following steps: displaying a visually detectable test spot on a surface display of the device, which surface display is viewed by the person by means of an eye of the person, wherein a spatial position of the head of the person remains unchanged with respect to the surface display; moving the test spot on the surface display by means of the device along a path during a measurement pass and activating an interaction means of the device by the person if the displayed test spot stops being visible or becomes visible again to the person at a current position during the movement of the test spot along the path, wherein, by activating the interaction means, the device is prompted to store the particular current position and any associated information from which it can be derived whether the test spot has stopped being visible or has become visible again for the person at the current position; and displaying a range of the visual field of the person on a further surface display, wherein said current positions form boundary points of the range.

The invention relates to a method for measuring the visual field of a person and a corresponding device.

Perimetry is an examination technique of the visual system. Human vision distinguishes between central and peripheral vision. Central vision can be determined as central optical acuity, peripheral vision is measured as peripheral visual field. The visual field corresponds to a hill, where the respective height of a point in the visual field corresponds to the visual acuity at this point. The maximum of this optical visual field hill lies above the retinal center, the fovea centralis. Towards the periphery, visual acuity continues to decrease. Peripheral visual acuity is measured using light difference sensitivity. Technically, there are two different methods to measure the visual field hill:
the kinetic perimetry
the static or profile or grid perimetry

Both methods attempt to define the visual field hill by having the patient signal the perception of the light stimulus. The perception threshold is thus determined subjectively. In kinetic perimetry, the test mark is advanced slowly centripetally in a hemisphere perimeter with constant brightness and then in further meridians of the entire circumference, whereas in static perimetry the brightness of the initially subthreshold test mark is gradually increased at fixed test spots located on a meridian. The perceived test patch is marked in each case. In kinetic perimetry, which scans the mountain horizontally, several isopters, lines of equal luminance difference sensitivity, are thus gradually created. In static perimetry, which scans the hill vertically, a hill profile is created running through the center of the visual field hill. Modern computer-controlled raster perimeters, which follow a predetermined test spot grid and also scan the visual field hill vertically, draw a map on which deviations from the norm are variously blackened.

The difference between perimetry and campimetry is that in campimetry the test objects are not offered in a hemisphere as in perimetry, but on a screen or a flat surface. Campimetry is therefore more suitable for measuring the paracentral visual field.

Common to all current methods is a slow horizontal or vertical approach to the perception threshold. This process is laborious and time-consuming. It must be taken into account that many patients are at an advanced age at the time of examination and that the willingness to cooperate can decline after a short time in many patients.

Current perimetry examinations usually require 15 minutes of concentrated cooperation per eye. The long examination time explains the difficulties in correctly characterizing the perceptual threshold at all measurement points. Therefore, the examination results vary greatly.

Computerized automated perimetry programs have relieved the examiner of the tedious examinations, but so far there is no relief for the patient.

Based on this background, it is the objective of the present invention to provide an improved method and device for measuring the visual field of a patient or person. This task is solved by the subject matter of the independent claims, preferred embodiments being stated in the corresponding dependent claims as well as in the description below.

According to claim 1, a method for measuring the visual field of a person by means of a device is disclosed, comprising the steps of:
a. displaying a visually detectable test spot on an area display of the device viewed by the person by means of an eye of the person, wherein a spatial position of the person's head with respect to the area display is fixed, and
b. Moving the test spot on the area display by means of the device on a predefined path during a measurement run and triggering an interaction device of the device during a measurement run, preferably whenever the displayed test spot becomes invisible to the person (so-called off point) or becomes visible again (so-called on point) during its movement on the path at a current position, wherein the triggering of the interaction device causes the device to store the respective current position as well as associated information from which it can be derived whether the test spot at the current position has become invisible to the person or visible again.

Preferably, the method comprises the further step of:
c. displaying information and/or an area (90) of the person's (P) field of view on the area display (2) and/or on a further area display, said current positions forming edge points of the area (90).

The method according to the invention is also referred to as rapid campimetry and represents a screening method with the aid of which the central 10° field of view of a person can be measured in the shortest possible time, e.g. within one minute. The comparatively large test spot density during the measurement run, which preferably lasts just under one minute, differs significantly from automated computer perimetry methods and facilitates the detection of scotomas. While the automated computer perimetry methods usually measure only at 16 or 25 test spots in the 10° visual field, the present method can efficiently measure the visual field of a person with high accuracy in a fine grid run, which scans, for example, the entire 10° visual field in each case on five paths above and below, left and right of the center of the examination field. Thus, the length of the path traveled by the test spot during a standard run of an examination is: distance patient to monitor*factor 0.5 to distance patient to monitor*factor 3, in particular distance patient to monitor*factor 1 to distance patient to monitor*factor 2, but in particular distance patient to monitor*factor 1.7625.

Example 01: 40 cm distance patient to monitor*factor 1.7625=70.5 cm distance

Example 02: 80 cm distance patient to monitor*factor 1.7625=141 cm distance

The relatively fast movement of the test spot and a preferably large contrast between a background of the area display and the test spot as well as the short examination time facilitate attention and increase the security of the measured data. Furthermore, the short examination time of e.g. 1 minute improves the applicability of the method in clinical routine. In the case of finding a visual field defect, an additional survey controlled by the examiner can ensure greater precision of the collected measurement data. The course of the nerve fibers is known. Assigned to the coordinates of the scotoma, the scotoma boundaries can be precisely defined programmatically according to the course of the nerve fibers in the direction of the optic nerve (see FIG. 3).

Preferably, the procedure is performed for each eye individually, covering the non-measured eye of the person. Said area of the visual field can be displayed, for example, by displaying or marking said current positions so that an impression of the area is created for the examiner. Furthermore, the area can be more clearly highlighted by creating an outer border line connecting the current positions on the further area display. Alternatively or additionally, it is possible to highlight the area within the displayed border line (or within a non-displayed virtual border line) in color against a background that is displayed on the further area display.

The path on which the test spot is moved does not necessarily have to be continuous, but can consist of individual sections separated from one another, which are then traversed, for example, one after the other. In principle, the path or the said sections can take all conceivable forms, wherein preferably the area of the person's field of vision to be examined is traversed several times by the test spot along the path, so that the area is scanned e.g. in two dimensions (e.g. horizontally and vertically, or in relation to the area displays, e.g., row-wise and column-wise).

According to an embodiment of the method, it is provided that the test spot has a speed S/f in [cm/s] when moving on the area display, where S/f is the ratio of the traveled distance S and a number f that is in the range from 2 to 7, in particular in the range from 3 to 6, in particular in the range from 4 to 5, wherein the number f is 4.7 in particular.

Example 01: Distance patient to monitor=40 cm; vertical sweep at 10° each above and below the center of the examination area totaling 14.1 cm on the screen, the calculation is then 14.1 cm/factor 4.7=3 cm/s.

Example 02: Distance patient to monitor=80 cm; vertical pass at 10° each above and below the center of the examination area totaling 28.21 cm on the screen, the calculation is then 28.21 cm/factor 4.7=6.00213 cm/s.

Furthermore, according to one embodiment of the invention, it is provided that during the measurement run within a period of 1 minute, at least 500 to 50000 different locations (or pixels of the area display) in the field of view are checked by means of the test spot, in particular at least 1000 to 25000 different locations (or pixels of the area display), in particular at least 1500 to 10000 different locations (or pixels of the area display), in particular at least 2500 different locations (or pixels of the area display). Preferably, during the measurement run, these locations, which are also referred to as test spots, are so close together that they partially overlap. With a traversed path of, for example, about 70.5 cm at a distance of 40 cm from the patient to the monitor, a dot or test spot trajectory is subjectively created, which, for example, with a narrow scotoma, results in only a brief darkening of the subjectively perceived trajectory in the scotoman area.

In particular, the invention is based on the basic idea that the test spot appears to the patient as a constant point of light that is moving. Furthermore, according to one embodiment of the invention, it is provided that the path has a total length of at least 17.625 cm to 705 cm, in particular of at least 35.25 cm to 352.5, in particular of at least 52.875 cm to 176.25 cm, in particular of at least 70.5 cm, wherein the test spot travels the entire path during the measurement process within a time period that is less than or equal to 1 minute.

Furthermore, in an embodiment of the method, it is preferably provided that the eye or eyes of the person, when moving the test spot along the path, has or have a distance to the area display that is in the range of 10 cm to 400 cm, preferably in a range of 20 cm to 200 cm, preferably in a range of 30 cm to 100 cm, the distance being in particular 40 cm.

According to a further embodiment of the invention, the path may have a plurality of mutually parallel first sections. According to a further embodiment, the path may have a plurality of mutually parallel second sections. In principle, this enables two-dimensional scanning of areas of the field of view or of the entire field of view of the person. In this context, it is preferably provided accordingly that the first sections cross the second sections, so that the first and second sections define, for example, a grid.

According to an embodiment of the method, the test patch is first moved along the first sections (e.g., each in a first direction) and then moved along the second sections (e.g., each in a second direction). Here, the first sections of the path can run vertically on the area display, with the second sections preferably running horizontally on the area display (or vice versa) Should one or both of the area displays have an inclination with respect to the horizontal, this is also referred to as vertical sections, which then run essentially perpendicular to the horizontal sections of the path.

Furthermore, according to one embodiment of the invention, it is provided that the path has at least one section that runs along the vertical or is inclined to the vertical. Furthermore, according to one embodiment, it is provided that the path has at least one section that runs in an arcuate shape, in particular in a semicircular shape. Furthermore, according to one embodiment of the invention, it is provided that the path has at least one section that crosses a nerve fiber and, in particular, runs orthogonally to the nerve fibers of the optic nerve of the eye of the person to be tested.

Furthermore, according to one embodiment, the path has a plurality of sections that are linear.

Furthermore, according to an embodiment of the invention, it is provided that for visualization of said area of the field of view, the current positions of the measurement run at which the test spot has become invisible are connected to a line which is displayed as a border line of the area on the further area display, and/or that the current positions of the measurement run at which the test spot has become visible are connected to a line which is also displayed as a border line of the area on the further area display.

According to a further embodiment of the method, it is provided that the area surrounded by the border lines is displayed on the further area display optically differentiable from the background of the further area display.

Furthermore, in an embodiment, the method according to the invention may provide that the detected area (limited by the stored current positions) is checked by repeatedly guiding the test spot, controlled by an examiner on the area display, out of said area into a surrounding area where the test spot is again visible to the person.

Furthermore, according to an embodiment of the method, it is provided that during the measurement run a central object is displayed on the area display, in particular in the form of a cross, which is weaker in light than the test spot, and which can be viewed by the person in order to fix a direction of gaze of the person (and thus of the field of view), the device preferably detecting during the measurement run whether a direction of gaze of the person deviates from the central object, and stopping the movement of the test spot if a deviation is detected.

Furthermore, according to an embodiment of the invention, it is provided that a movement of the test spot during the measurement run is coupled to the interaction device in such a way that when the interaction device is triggered, a stop and a return movement of the test spot (in particular corresponding to the reaction time of the person) along the path in the opposite direction is performed.

According to an embodiment of the invention, it is provided that the test spot has a brightness that is particularly in the range of 200 cd/m² to 1000 cd/m², particularly in the range of 300 cd/m² to 400 cd/m².

Preferably, according to an embodiment, the test spot is displayed on the area display in front of a (preferably dark) monotone background displayed on the area display, wherein the luminance of the test spot is greater than the luminance of the background. For example, the background can be displayed in dark blue, especially with a hue of 000066 (hexadecimal RGB code). The background can also be in another dark color.

Furthermore, according to an embodiment, the central object (e.g. cross) may have a hue with the hexadecimal RGB code 1111AA. The test spot can have a hue with the hexadecimal RGB code FFFFFF. However, other color or contrast combinations are also conceivable.

According to an embodiment of the invention, the test spot on the area display is moved within an inspection field (i.e., said path is located in the inspection field and, in particular, defines the edges thereof), the inspection field having four corners as well as a height H.

Furthermore, according to an embodiment of the invention, it is provided that the test spot is generated on the area display in such a way that it has a diameter in the corners of the inspection field which is in the range from H/4.7 to H/470, in particular in the range from H/20 to H/200, in particular in the range from H/30 to H/100, the diameter being in particular H/47, where H in each case is the height of the inspection field in cm in the vertical direction.

Furthermore, according to an embodiment of the invention, the diameter of the test spot changes automatically in relation to a distance from the central object, with the diameter decreasing towards the central object and having a minimum diameter in the region of the central object.

Preferably, the minimum diameter of the test spot is in the range of H/18 to H/1800, in particular in the range of H/75 to H/750, in particular in the range of H/125 to H/500, where in particular the minimum diameter is H/180, where H is in each case the height of the examination field in cm in the vertical direction.

In an embodiment of the method according to the invention, the steps described above are preferably each carried out automatically, except for the triggering of the interaction unit by the person, which is effected by an action of the person. Furthermore, if necessary, a tracking of the test spot can be performed manually by the examiner in order to accurately sound out any limits at which the visibility of the test spot changes, if necessary. The processing unit may be constituted by (or comprise) a computer on which may be executed a computer program comprising instructions that cause the processing unit to perform the method steps (using the further components of the device). Insofar as reference is made to a processing unit, this also includes embodiments in which the method steps are carried out by a plurality of cooperating processing units. Instead of at least one computer or at least one processing unit on which a computer program is executed, the processing unit can also be formed by a hard-wired control unit.

Furthermore, according to an embodiment of the method, it is provided that the speed of the test spot is temporarily slowed down significantly, e.g. by at least 50%, if required by the investigator, in particular by interaction with a user interface of the device, in particular in order to specify a current position at which the test spot has become invisible or has become visible again (e.g. when said position is traversed again with the test spot if required).

Furthermore, according to an embodiment of the method, it is provided that after the first measurement run along the path described above, when detecting current positions at which the test spot has become invisible or has become visible again, in each case at least one further measurement run is carried out at the respective stored current position, particularly in order to be able to detect or visualize said area (see above) more precisely.

Furthermore, according to an embodiment of the method, the method further comprises the step of: performing a further measurement run for each section of the path of the (first) measurement run which extends between two adjacent stored current positions, in which in each case the test spot on the area display is moved by means of the device along a further path within an area on the area display which contains the respective section as a center, and in each case triggering of the interaction device of the device by the person, when the displayed test spot becomes invisible to the person at a current position or becomes visible again during its movement along the further path within the area, wherein the triggering of the interaction device causes the device to store the respective current position of the test spot in the area and associated information from which it can be derived whether the test spot has become invisible to the person at the current position or has become visible again.

According to a further embodiment of the method, it is provided that after the respective further measurement run, further measurement runs are carried out for sections of the further path found in the respective area, which in each case extend between two adjacent stored current positions of the further path and lie at an edge of the area, until in the process a section is found which was previously found in the measurement run or in a further measurement run, wherein for this section a further measurement run is likewise carried out, in which in turn in each case the test spot on the area display is moved by means of the device along a further path within an area on the area display, which contains the section as a center, the interaction device of the device being triggered in each case by the person if the displayed test spot becomes invisible to the person at a current position or becomes visible again during its movement along the further path within the area, the triggering of the interaction device causing the device to store the respective current position of the test spot in the area and associated information from which it can be derived whether the test spot has become invisible to the person at the current position or has become visible again.

According to an embodiment of the method, it is further provided that the respective area has a size of 5°, in particular 2.5°, in particular 1° around the center of the area, the distance of adjacent parallel sections of the further path in the area during the respective further measurement run to each other being in each case 2.5°, in particular 1°, in particular 0.5°.

By optionally storing measurement results of other persons, e.g. in a database, Artificial Intelligence (AI) can be used to identify further potential visual field failure areas of a person. In particular, two basic AI methods can be applied for this purpose:
a statistical analysis based on the stored current positions (e.g. in the coordinate system of the area display or further area display, where the respective position is specified as an (x,y) coordinate pair)—e.g. using AI Machine Learning, or
An image analysis of the visualized examination results or areas—e.g. using AI Deep Learning.

Based on the results of AI analysis of other individuals and the likelihood of visual field loss compared to other individuals, additional areas can then be automatically checked for potential additional visual field loss not yet detected.

Accordingly, according to a further embodiment of the method, the method comprises the further step of automatically selecting at least one area by means of an AI algorithm that has been trained with a plurality of data sets of different persons, wherein a further measurement run is carried out in which in each case the test spot on the area display is moved by means of the device along a further path within the area on the area display, wherein in each case the interaction device of the device is triggered by the person if the displayed test spot becomes invisible to the person at a current position or becomes visible again during its movement along the further path within the area, wherein the triggering of the interaction device causes the device to store the respective current position of the test spot in the area as well as associated information from which it can be derived whether the test spot has become invisible to the person at the current position or has become visible again.

Furthermore, according to an embodiment of the method, it is provided that the AI algorithm is designed to select the at least one area based on data records of different persons, wherein the respective data set of a person includes the stored current positions of the person, or wherein the AI algorithm is designed to select the at least one area based on data sets of different persons, wherein the respective data set corresponds to the visualized area of a person.

Furthermore, according to an embodiment of the method, during a manual area examination, the examiner marks areas on the examination area in which further tests are carried out automatically.

According to an embodiment of the method, the method further comprises the step of: performing at least one further measurement run for an area selected by the examiner, wherein the test spot on the area display is moved along a further path within the area on the area display by means of the device, and in each case triggering the interaction device of the device by the person, when the displayed test spot becomes invisible to the person at a current position or becomes visible again during its movement along the further path within the area, the triggering of the interaction device causing the device to store the respective current position of the test spot in the area and associated information from which it can be derived whether the test spot has become invisible to the person at the current position or has become visible again.

Furthermore, according to an embodiment of the method, it is provided that the examiner performs a manual free examination in which the test spot is controlled completely manually on the examination area (e.g., area display or further area display) by the examiner.

In this respect, it is further provided according to one embodiment of the method that the method further comprises the step of: Performing at least one further measurement run, wherein the test spot on the area display is moved along a further path under the control of the examiner by means of the device, and in each case triggering of the interaction device of the device by the person when the displayed test spot becomes invisible to the person during its movement along the further path within the area at a current position or becomes visible again, wherein the triggering of the interaction device causes the device to store the respective current position of the test spot in the area as well as associated information from which it can be derived whether the test spot has become invisible to the person at the current position or has become visible again.

When displaying said area of the person's field of view on the area display and/or on a further area display, the current positions stored in the further measurement runs now also form edge points of said area. The accuracy of the area is thereby increased accordingly. In particular, for the visualization of the area, the current positions stored in the measurement run as well as in the further measurement runs and at which the test spot has become invisible, can be connected with a line which is displayed as a border line of the area on the further area display. Alternatively or additionally, the current positions stored during the measurement run and the further measurement runs at which the test spot has become visible can be connected to a line that is displayed as a border line of the area on the further area display, whereby in particular the area surrounded by the border lines is displayed on the further area display in a way that is visually distinguished from the background of the further area display.

During the measurement runs or further measurement runs described above, the examiner can optionally influence the respective measurement run in each case, e.g. by
a variable test spot size (e.g. start and end size of the test spot as well as a change of the test spot size),
a speed of the test spot along the respective path,
a direction of the path to be traversed (e.g. horizontal, vertical, diagonal)
a number of sections of a path to be traversed on the area display or further area display or in an area.

Another aspect of the invention relates to a computer program comprising instructions which, when the computer program is executed by a computer, cause the computer or device to perform the steps of the method of the invention.

According to a further embodiment of the method according to the invention, it is provided that, in particular during the measurement run, a content of the area display is transmitted to the further area display via a data transmission link and/or that (in particular during the measurement run) a content of the further area display is transmitted to the area display via a data transmission link.

In the above-mentioned embodiments, the data transmission link may be a computer network connection, in particular an Internet connection, which may use one or more known network protocols. The data transmission link may also be a radio link or may be formed in sections by a radio link.

According to a further embodiment of the method, it is provided that the device comprises a processing unit, in particular for displaying and moving the visually detectable test spot on the area display.

According to a further embodiment of the method, it is provided that the processing unit is a local processing unit (client) at the location of the person, wherein the respective current position as well as the associated information are stored on the local processing unit and are transmitted to a further processing unit via a data transmission link and are evaluated on the further processing unit, generating a result data set.

According to an alternative embodiment of the method, the processing unit is a local processing unit (client) at the location of the person, the respective current position and the associated information being stored on the local processing unit and being evaluated on the local processing unit to generate a result data set, the result data set being transmitted to a further processing unit via a data transmission link.

The local processing unit can be a computer or client of the person, in particular in the form of a desktop computer, a laptop or a tablet PC. The further processing unit can be a (remote) server.

According to a further alternative embodiment of the method, it is provided that the device further comprises a local processing unit at the location of the person, which is connected to the area display, wherein the processing unit (in particular server) causes the display and movement of the visually detectable test spot on the area display via a data transmission link to the local processing unit, wherein the respective current position as well as the associated information are stored on the processing unit (server) and evaluated to generate a result data set.

The local processing unit may in turn be a computer or client of the person (see above). The processing unit is preferably a (remote) server that is connected to the local processing unit via a data transmission link.

In the above-mentioned embodiments, the data transmission connection may be a computer network connection, in particular an Internet connection, which may use one or more known network protocols. The data transmission link may also be a radio link or may be formed in sections by a radio link.

The result data set may have graphically representable data corresponding to or encoding said area of the subject's visual field.

Another aspect of the present invention relates to a computer program comprising instructions that, when the computer program is executed on the processing unit, cause the processing unit or the device to perform the steps of the method according to any one of claims 22 to 25.

Another aspect of the invention relates to a computer-readable storage medium on which the computer program according to the invention is stored.

A further aspect of the invention relates to a device for measuring the field of view of a person, which is in particular arranged to carry out the method according to the invention (to this extent, the device can also be further characterized by the individual features of the method according to the invention described herein). The device comprises at least:
an area display configured to be viewed by the person,
a processing unit configured to display a test patch on the area display to the person and move it along a predefinable path on the area display,
an interaction device configured to be triggered by the person when the displayed test patch becomes invisible to the person or becomes visible again at a current position during its movement on the path, wherein the processing unit is configured to store the respective current position and associated information from which it can be derived whether the test patch has become invisible to the person or visible again at the current position when the interaction device is triggered.

In particular, the device may be configured to perform or be used in performing the steps of the method of any one of claims 1 to 25.

According to one embodiment of the invention, the interaction device comprises an actuating element for triggering the interaction device. This may be, for example, one of the following actuating elements: a switch, a microphone, a touch screen, a rotary knob, a slider.

If the area to be examined or the field of view is scanned by the test spot in such a way that the test spot becomes alternately invisible and visible again, it is sufficient if the interaction device is triggered with a single signal that is generated, for example, by actuating one of the above-mentioned actuating elements once when the visibility of the test spot changes. In this respect, a microphone is also understood to be an actuating element. A signal from the interaction device can reach the processing unit in various ways and be received and processed by it. Conceivable here are, for example, electrical signals, electromagnetic signals, optical signals, acoustic signals.

Furthermore, according to an embodiment of the device, it is provided that the device is configured to generate a central object (in particular in the form of a cross) on the area display, which is observable by the person by means of the eye to be measured. According to a further embodiment of the device, it is provided that the device comprises a further area display configured to be viewed by an examiner.

Furthermore, according to one embodiment of the device, it is provided that the area display is formed by or comprises a screen.

Furthermore, in one embodiment of the device, the further area display for the examiner is formed by a screen or comprises a light source for generating an image on a projection surface.

According to a further embodiment of the device, the processing unit is further configured to determine and display to the examiner on the further area display an area of the person's field of view, wherein said detected or stored current positions form edge points of the area.

According to a further embodiment of the device, it is provided that the device comprises a fixation unit configured to fix a head position of the person with respect to the area display.

According to a further embodiment of the device, it is provided that the device comprises a further processing unit, wherein the processing unit is configured to transmit the current positions as well as the associated information to the further processing unit via a data transmission link, wherein the further processing unit is configured to evaluate the current positions as well as the associated information to generate a result data set.

The result data set may have graphically displayable data corresponding to or encoding said area of the person's field of view. The area display and interaction device may be connected to the processing unit, which is provided or positionable at the person's location. The further processing unit may be a remote server. The data transmission link may be a computer network connection, in particular an internet connection, which may use one or more known network protocols. The data transmission link may also be a radio link, or may be formed in sections by a radio link. Further, the further area display may be connected to the server or to a client connected to the server via a computer network connection. Thus, the above-described embodiment allows a patient to perform the measurement run at his local area display at home and send the corresponding data (current positions and related information) to a remote server for evaluation.

According to a further embodiment of the device, it is provided that the device comprises a further processing unit, wherein the processing unit is configured to evaluate the current positions and the associated information to generate a result data set and to transmit the result data set to the further processing unit via a data transmission link.

The result data set may in turn have graphically displayable data corresponding to or encoding said area of the person's field of view. The area display and interaction device may be connected to the processing unit, which is provided or positionable at the person's location. The further processing unit may be a remote server. The data transmission link may again be a computer network connection, in particular an internet connection, which may use one or more known network protocols. The data transmission link may also be a radio link, or may be formed in sections by a radio link. Further, the further area display may be connected to the server or to a client connected to the server via a computer network connection. The present embodiment also enables the patient to perform the measurement run at his local area display at home, wherein an evaluation of the measurement data (current positions and associated information) is also performed locally here and only the result data set is transmitted to the remote server or to the examiner.

According to a further embodiment of the device, the device further comprises a local processing unit at the location of the person, which is connected to the area display, wherein the processing unit (in particular server) causes the display and movement of the visually detectable test spot on the area display via a data transmission link to the local processing unit, wherein the respective current position as well as the associated information are stored on the processing unit (server) and evaluated to generate a result data set.

Again, the result data set may have graphically displayable data corresponding to or encoding said area of the person's field of view. The area display and interaction device may be connected to the local processing unit, which may be provided or positioned at the person's location. The processing unit may be a remote server. The data transmission link may be a computer network connection, particularly an Internet connection, which may use one or more known network protocols. The data transmission link may also be a radio link, or may be formed in sections by a radio link. Further, the further area display may be connected to the server or to a client connected to the server via a computer network connection. The embodiment described above thus enables the patient to perform the measurement run at his local area display at home. The measurement run is thereby performed and evaluated on the server. The patient can, for example, access an Internet page via his local processing unit using a web browser, which forms a user interface for performing the measurement run. A corresponding application can be executed on the server.

Furthermore, the device according to the invention can be further characterized by the features described above in connection with the method.

Area displays are characterized in particular by the fact that they have essentially flat surfaces for the graphic display.

In the following, embodiments of the present invention and further features and advantages of the invention will be explained with reference to the figures. It is shown in

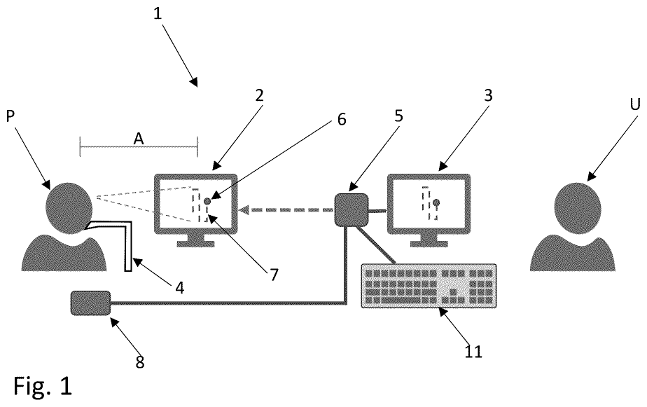

FIG. 1 a schematic representation of an embodiment of a device according to the invention,

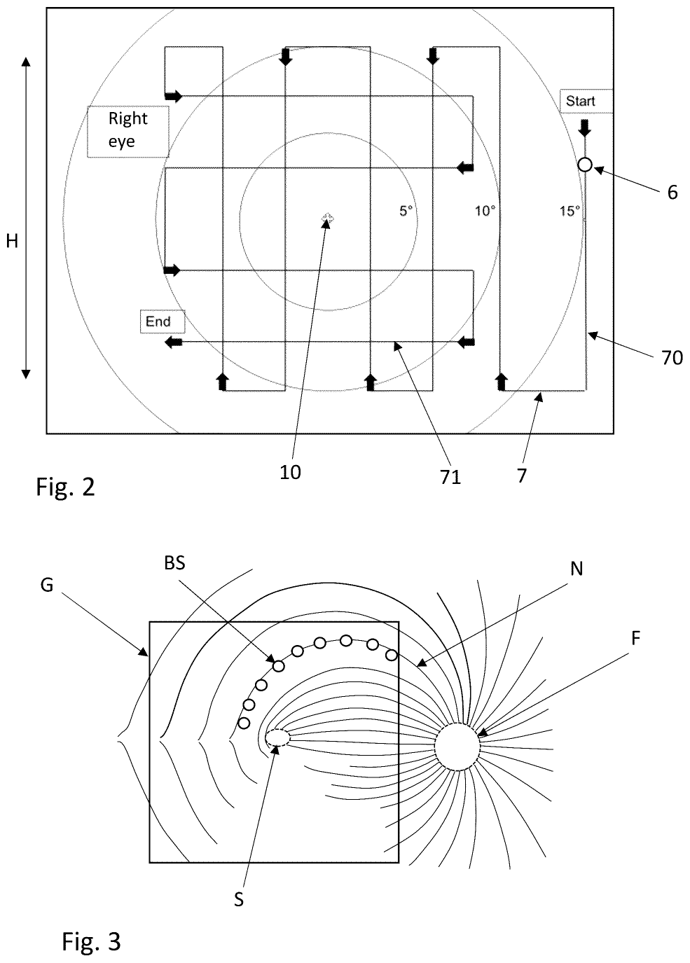

FIG. 2 an illustration of a path that is traced from the test spot in a method according to the invention,

FIG. 3 a schematic representation of the optic nerves for comparison with the sections of the pathway shown in FIG. 2,

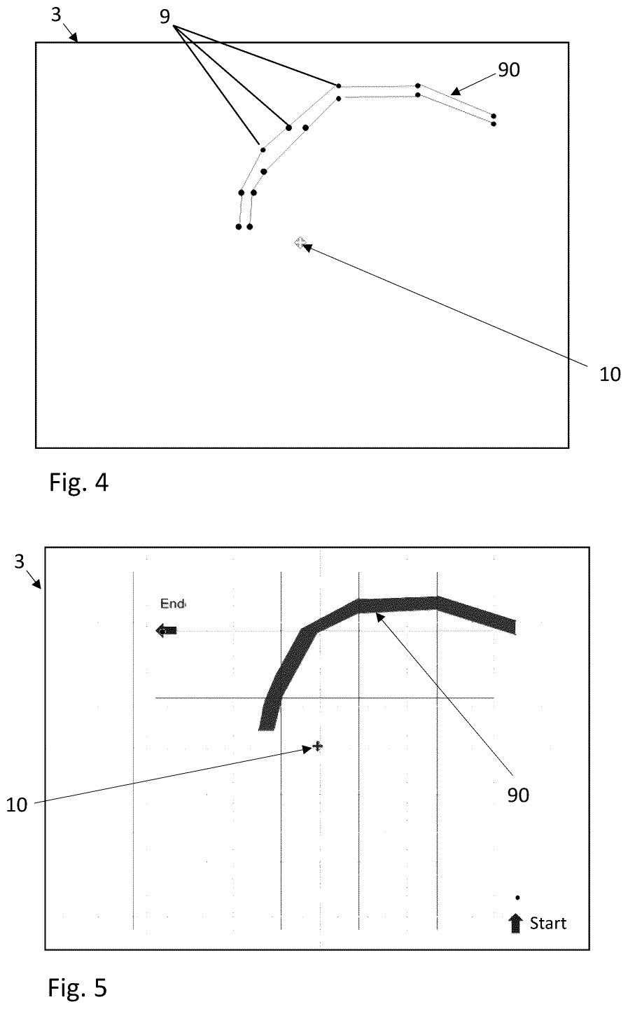

FIG. 4 a representation of the detected or stored current positions of the test spot at which it changes its visibility,

FIG. 5 a colored highlighting of the area of a person's visual field measured with a method or device according to the invention according to FIG. 2, which may correspond to a visual field defect,

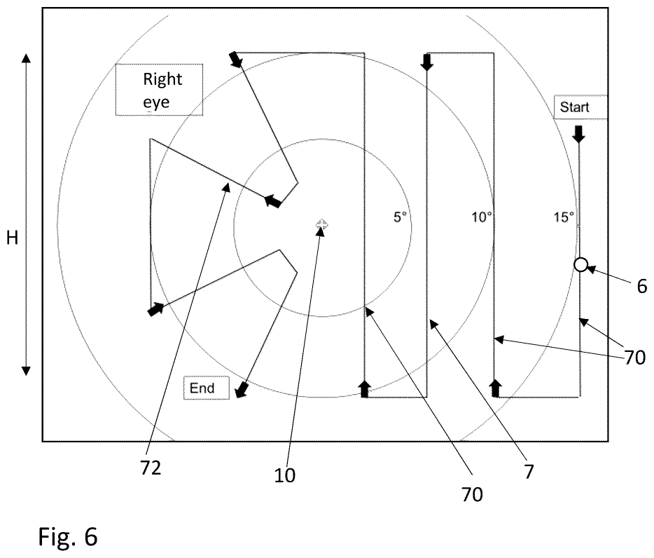

FIG. 6 an illustration of a further path that can be scanned by the test spot in a method according to the invention,

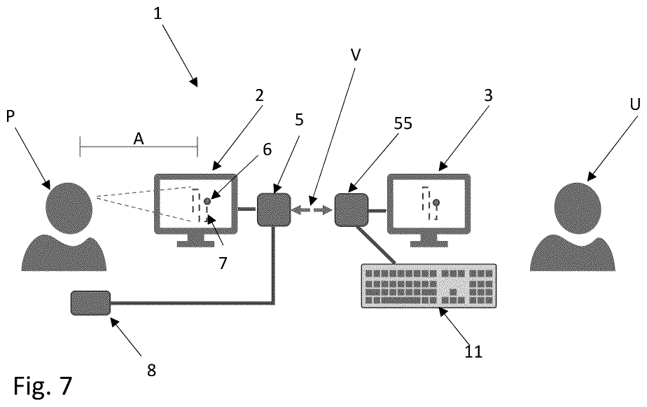

FIG. 7 a schematic representation of an embodiment of a device according to the invention, which is particularly suitable for telemedical purposes,

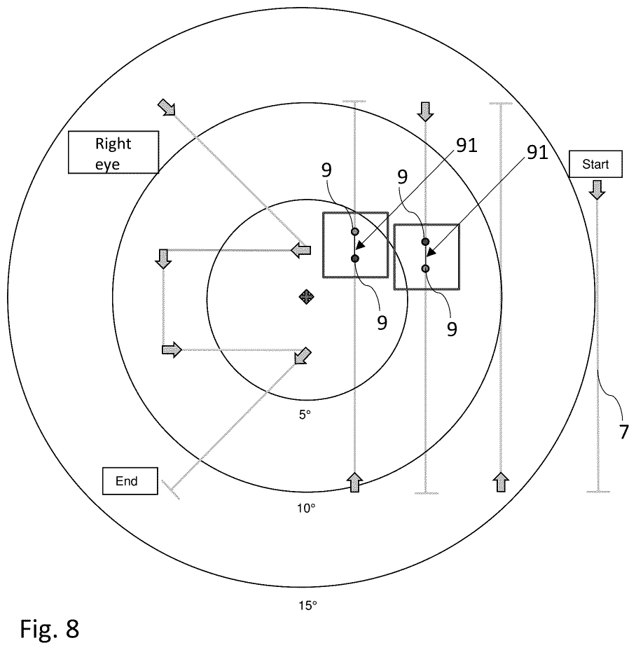

FIG. 8 a schematic representation of the current positions recorded during a first measurement run at which the test spot has become invisible or visible again,

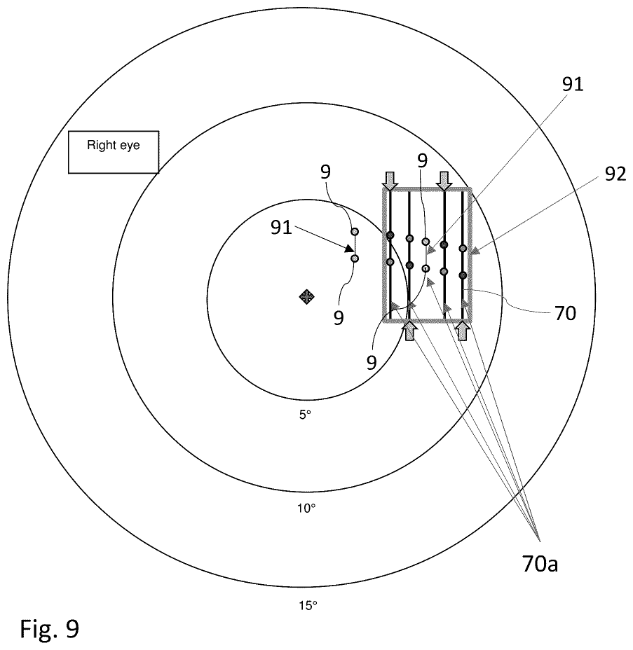

FIG. 9 a schematic representation of a further measurement run in a reduced area whose center corresponds to a loss previously detected during a first measurement run,

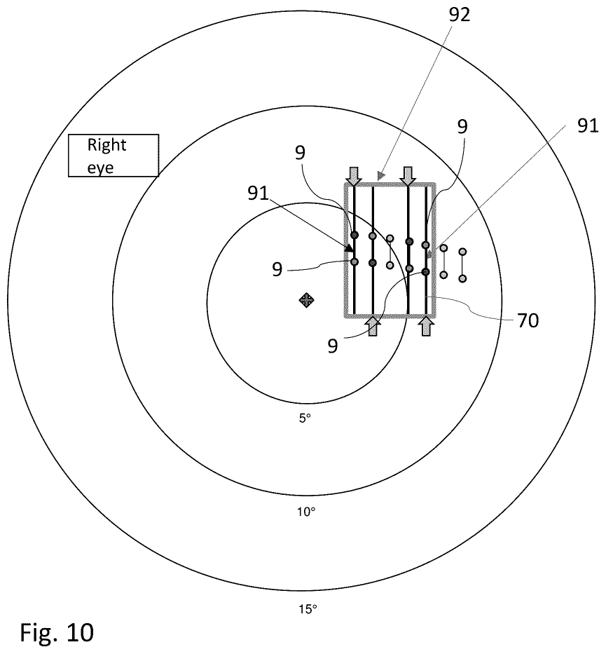

FIG. 10 a schematic representation of another measurement run in a reduced area whose center corresponds to a loss previously detected in an area,

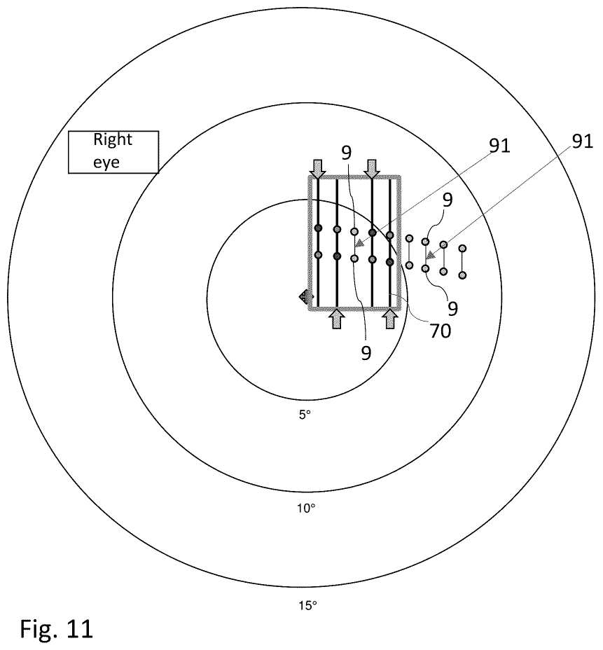

FIG. 11 a schematic representation of a further measurement run in a reduced area whose center corresponds to a further loss detected in the first measurement run,

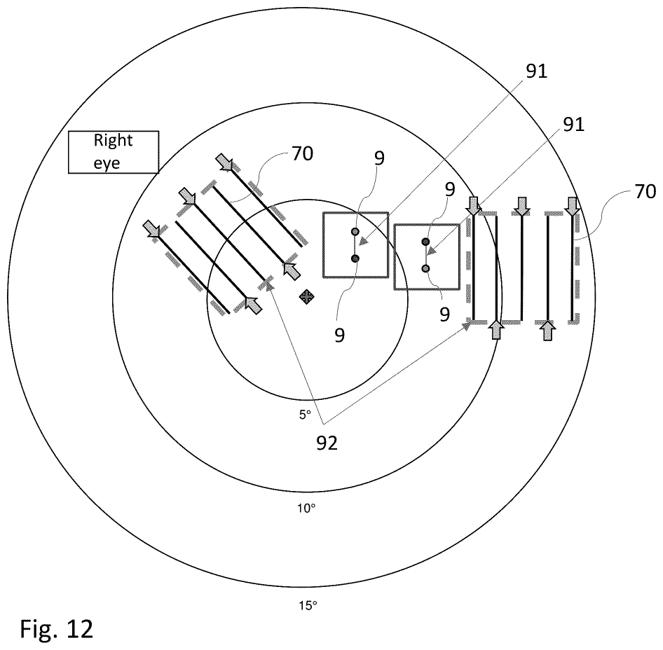

FIG. 12 a schematic representation of further measurement runs in two reduced areas selected by means of an AI algorithm trained using patient data,

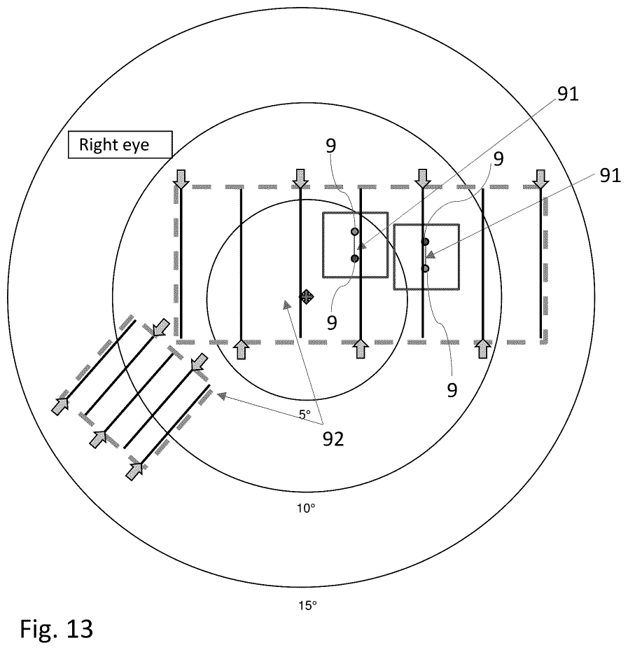

FIG. 13 a schematic representation of further measurement runs in two reduced areas previously defined by the examiner, and

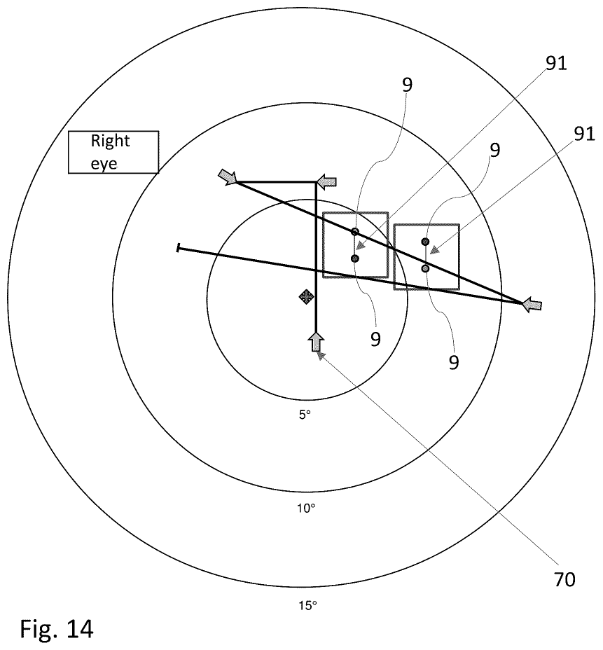

FIG. 14 a schematic representation of a further measurement run, in which the test spot is moved along a further path, which is determined e.g. in real time by the examiner.

The method according to the invention is also referred to in the following as rapid campimetry (RAP CAMP or RAP-CAMP). In the method, according to one embodiment, e.g. using a device according to FIG. 1, which is described in detail below, basically the following steps are performed:
Displaying a visually detectable test spot 6 on an area display 2 of the device 1 viewed by the person P by means of an eye of the person P, wherein a spatial position of the head of the person P with respect to the area display 2 remains unchanged,
Movement of the test spot 6 on the area display 2 by means of the device 1 along a path 7 during a measurement run and triggering of an interaction device 8 of the device 1 by the person P when the displayed test spot 6 becomes invisible or visible again to the person P during its movement along the path 7 at a current position 9 (cf. FIG. 4), wherein the device 1 is caused by the triggering of the interaction device 8 to store the respective current position and associated information from which it can be derived whether the test spot 6 has become invisible to the person P at the current position or has become visible again, and
Displaying an area 90 of the person's P field of view on another area display 3, said current positions 9 forming edge points of the area 90.

This can directly result in decisive advantages of the process according to the invention or individual embodiments of the process:
A duplication of the test spots 6 and acceleration of the examination.
In the 10° range of the field of view, inspection can be performed at several hundred to several thousand locations instead of 16 or 25 (as with modern computer perimeters), improving the detectability of absolute scotomas.
Concentration and cooperation with the patient are easy to practice for about 1 minute approx. This makes the patient's information more reliable.
The applicability of the method increases, since about 1 minute can be integrated into a routine examination, in contrast to perimetry
the RAP-CAMP becomes an easily applicable screening method for absolute scotomas.
Every patient with suspected normal tension glaucoma can be examined. Approximately 0.3% of the population over 40 years of age in Germany has normal tension glaucoma.
Risk factors for normal tension glaucoma are myopia and migraine. Migraine incidence in Germany is about 1%. Every patient over 40 years of age with myopia and migraine should be examined. The frequency of diagnosis of normal tension glaucoma will increase, because scotomas can be found faster and more reliable and more patients will be examined.
Glaucoma patients (about 1% of the population over 40 years of age in Germany) can easily be controlled more frequently.
However, the method described herein is in particular not a diagnostic method, but only a measurement method, which in particular does not include a diagnostic step, but in particular only performs a measurement of the visual field.

According to an embodiment, the method according to the invention can be carried out, for example, with the following steps:
The patient sits at a distance of e.g. 40 cm A in front of the area display 2 in the form of a screen.
The head of patient P is fixed in place by a fixation unit 4 comprising a chin and forehead support.
A bright white test spot 6 runs across the otherwise dark screen 2 in rapid succession.
The test spot 6 passes through the field of view horizontally, vertically and diagonally, so that a grid of quadrants is covered by the test procedure.
As soon as the test spot 6 becomes invisible to patient P and later becomes visible again, patient P signals the change in each case.
At the end of the examination, all such detected current positions 9 of the test spot, at which the test spot 6 became invisible (off-points) or became visible again (on-points), are connected with each other in a preliminary connection in such a way that an area 90 of the visual field, which may correspond to a visual field failure, becomes recognizable (cf. FIG. 4).
The visual field loss can subsequently be specified during kinetic perimetry.

FIG. 1 shows an embodiment of a device 1 for measuring the visual field of a person P, which is suitable for carrying out the method according to the invention. The device 1 comprises an area display 2, which is configured to be viewed by the person P. The device 1 further comprises another area display 3 configured to be viewed by an examiner U, and optionally a fixation unit 4 configured to fix a head position of the person P with respect to the area display 2. The device 1 further comprises a processing unit 5 (e.g., in the form of a computer) configured to display and move a test spot 6 on the area display 2 of the person P along a predefinable path 7 on the area display 2. The device 1 further comprises an interaction device 8 configured to be triggered by the person P when the displayed test patch 6 becomes invisible to the person P or becomes visible again during its movement along the path 7 at a current position 9, wherein the processing unit 5 is configured to store, when the interaction device 8 is triggered, the respective current position 9 as well as associated information from which it can be derived whether the test patch 6 has become invisible to the person P or has become visible again at the current position 9 (cf. FIG. 4). The processing unit 5 is further configured to display an area 90 of the field of view of the person P on the further area display 3, wherein said current positions 9 form edge points of the area 90.

The fixation unit 4 can have, for example, a chin and forehead support on which the patient P can place his chin. In front of this, a holder can be provided for the possible use of any spectacle lenses that may be required.

Preferably, the area display 2 is designed in the form of a screen and is arranged at a fixed distance A from the uncovered eye of the patient P. The interaction device 8 can be operated by the patient, e.g., by making sings/voicing.

Furthermore, a fixation control unit may be provided to ensure that the patient P fixates a central object 10 (e.g., a cross) with the eye to be examined. Preferably, the observation of this central fixation cross 10 is controlled by the examiner U and precisely adjusted at the beginning of the examination (e.g., by corneal reflex or pupil image). In case of deviation from the central fixation 10, an automatic stop of the test spot run is preferably performed by the fixation control unit.

The area display 2 or screen 2 is preferably completely different from any ordinary perimetry and campimetry. It preferably has the following characteristics:
The area display 2 is darkened and has, for example, a dark blue hue; other dark colors are also possible.
The distance of the patient's eye P to the area display or screen 2 is set to 40 cm, for example. Accordingly, the campimetry according to the invention is performed in the central 10° range at 14.1 cm grid size (vertical) to 15° at 21.4 cm grid size (horizontal). In this range lie all early cases of glaucoma and also the blind spot F (cf. FIG. 3), which can show the patient the disappearance of the test spot.

FIG. 3 shows schematically the course of the nerve fibers N at the back of the eye. The square corresponds to the central 10° visual field G. Furthermore, the location S of the sharpest vision is shown. The circles BS show exemplarily the course of an arcuate scotoma.

According to an example of the invention, the test spot 6 is bright white. Unlike in the hemisphere perimeter, there is no addition of the luminance (environment+test spot). Instead of the dark background, there is the bright test spot.

The central object 10 (e.g. the central cross) serves as the fixation point, see above. It is therefore preferably clearly visible to allow easy observation, but on the other hand should not be too bright and dazzle the patient P. It is therefore preferably weaker in light than the test spot 6 and can have, for example, a light blue hue (other colors are also possible).

The size or diameter of the test spot 6 is preferably variably adjustable and can be adapted as required with regard to the patients being examined. According to one example, the diameter of the test spot may be 2 mm corresponding to a range of 0.3° in the field of view. For certain applications, a test spot of 2.5 mm (range of 0.4° in the field of view) may be necessary with the test area more than 10° from the center, as visual acuity decreases toward the periphery. Also, patients with poor visual acuity should be able to be examined. A test spot as small as 1 or 1.5 mm would allow more precise examination as long as it is easily perceived. However, the examiner may be larger. Preferably, the device 1 is designed in such a way that the test spot 6 becomes larger with increasing distance from the center 10.

One of the significant differences to all prior art visual field examination methods is the running speed of the test spot 6. This is because it moves comparatively fast through the visual field of the patient 6. The perceptibility of the test spot 6 can therefore be measured at a very large number of locations in a short time. According to one example of the invention, for example, it is possible to check at 3750 locations in the 10° range in 30 seconds. Modern computer perimeters test at 16 to 25 points in the 10° range, depending on the program. The inspection speed of the present invention, on the other hand, may be, for example, 125 frames per second. The running speed of the test spot 6 can be adjusted in the method or device 1 of the invention based on the individual requirements of the patients being examined.

FIG. 2 illustrates a possible path 7 that the test spot 6 travels during a measurement run due to a corresponding configuration or design of the device 1.

According to an embodiment of the invention, the device 1 is designed to run the test spot 6 along a path 7, first from right to left along six vertical sections 70, and in particular alternating from top to bottom as well as from bottom to top, and then along four horizontal sections 71. The length of the path 7 may be, for example, about 130 cm on the area display 2. If a speed of the test spot of 3 cm/s is used, the example results in an inspection time of less than 1 minute.

FIG. 5 shows an alternative configuration of the path 7. Here, the test spot 6 is first guided from right to left along four vertical sections 70, in particular alternating from top to bottom and from bottom to top. Hereafter, the test spot 6 is guided along four sections 72 which are inclined with respect to the vertical, crossing, for example, the 5° circle and/or the 10° circle, in particular orthogonally to the respective circle. This proves to be advantageous, since the nerve fibers N of the optic nerve (cf. FIG. 3) are thereby cut as perpendicularly as possible.

The further area display 3 or the further screen 3 of the examiner U preferably also has a central object/cross 10 which corresponds to the patient's fixation point 10 on the screen 2. Furthermore, the 5°, 10° and 15° circles for the dimension of the visual field are preferably displayed.

The two area displays/screens 2, 3 are coupled with each other in particular and cooperate, wherein the test spot 6 also runs visibly for the examiner U over the further screen 3. If the patient P signals the disappearance or reappearance of the test spot 6, then these spots or current positions 9 (cf. FIG. 4) are marked on the screen 3 of the examiner U.

At the end of the examination, all positions or points 9 at which the test spot 6 became invisible (off-points) or became visible again (on-points) are connected with each other by the processing unit 5 of the device 1 in a preliminary connection in such a way that a probable form of the visual field loss becomes recognizable, as it is exemplarily shown in FIG. 4. The pre-drawn probability of visual field loss can be verified, if necessary, as in kinetic perimetry, by moving the test spot (cursor like test mark) from the region of invisibility to the region of visibility. This test spot movement controlled by the examiner U is controlled by the examiner U e.g. via an input means 11, in particular direction keys of the computer or processing unit 5. The device 1 or processing unit 5 can further be designed to automatically visualize said area 90 or the suspected visual field loss at the end of the examination, as shown in FIG. 5.

During the measurement run, the patient P can signal via various means that the visibility of the test spot 6 has changed, e.g. by pressing a button, speaking or the like. The interaction device 8 provides a corresponding input means for this purpose.

The position 9 of the losses signaled by patient P is stored by device 1. After a first measurement run, the areas signaled by patient P can be specified manually by the examiner. Such a specification of the presumed visual field failure 90 is performed, for example, by approaching more slowly again the approximate location of the change in visibility after the first measurement run and focusing on the failure areas signaled by the patient P. In principle, the method according to the invention also exhibits suitability for design as a telemedical method. For example, the measurement run or the examination can be performed via the Internet. A patient P can be examined if he only has a computer in his premises, and the examiner U can perform the measurement run remotely if required. Especially for experienced patients, e.g. glaucoma patient, who have been examined several times, know the examination procedure, which could therefore be repeated autonomously if necessary (cover one eye, keep distance of e.g. 40 cm, fix central object 10, e.g. press button when examination spot disappears). Auxiliary personnel would not be required. For example, a patient could collect his findings at regular intervals and send them to his doctor (examiner U). In other parts of the world, where perimeters are not available, a support person, who is instructed in the examination technique via the Internet, could collect and send campimetry findings.

FIG. 1 shows a further embodiment of a device 1 for measuring the visual field of a person P, which is suitable for carrying out the method according to the invention and, moreover, does not require an examiner U to be present at the location of the patient P.

In this context, the device 1 comprises an area display 2 configured to be viewed by the person P. The area display (in particular screen) 2 is connected to a local processing unit (e.g. in the form of a computer) 5, which in turn is connected to an interaction device 8, which may be, for example, a keyboard or a microphone of the computer. The local processing unit 5 is connected via a data transmission link V (e.g. an Internet connection) to a further processing unit 55, which may be, for example, a server. On the server side, a further area display 3 and a keyboard 11 for an examiner U may be provided. With regard to the measurement run, it is now possible to implement this by means of a computer program which is executed on the local processing unit 5. On the local processing unit 5, the measurement data (current positions and associated information) can be stored and evaluated while generating a result data set. The result data set can be transmitted to the server 55 or to the investigator U via the data transmission link V. Alternatively, it is also possible to transmit the measurement data to the server 55 and evaluate it there to generate the result data set.

Furthermore, it is possible that the measurement run is realized by means of a computer program which is executed on the further processing unit 55 (e.g. server), e.g. as a browser-based application. The patient can access this application via the data transmission link or Internet connection V and the measurement data collected during the measurement run can be stored on the server 55 and evaluated there to generate the result data set.

In the aforementioned process variants, the result data set may have graphically representable data corresponding to or encoding said area 90 of the person's visual field (cf. FIGS. 4 and 5). This area 90 can be displayed to the patient, for example, via the area display 2 or to the remote examiner U via the further area display 3.

The examiner U can communicate with the patient P, in particular during the measurement run. Such (e.g. audiovisual) communication between the patient and the examiner (e.g. in the form of a video chat) can also be realized via a data transmission link V between the two processing units 5, 55 or by other means.

The various designs of the embodiment according to FIG. 5 thus enable the patient to carry out the measurement run on his local area display at home. The patient can be guided or accompanied by the examiner in real time if necessary.

Furthermore, according to an embodiment of the method according to the invention or the corresponding device, it is provided that after the first measurement run along the defined path described above, when a visual field loss is detected (i.e., in the event that current positions 9 are detected at which the test spot becomes invisible and then visible again), further, optional detailed examinations or further measurement runs can take place that scan any detected visual field losses more precisely. This can be done in the manner described above (e.g. for a smaller area 92), in particular (partially) automated (cf. FIGS. 9 to 11), and further with the support of artificial intelligence (AI)-based methods (cf. FIG. 12). Furthermore, a manual area examination (cf. FIG. 13) or even a manual free examination (cf. FIG. 14) can be performed.

In detail, for example, sections 91 along the path 7 detected during the first measurement run or standard run, which extend between two successive current positions 9 at which the test spot 6 becomes invisible to the examined person P or becomes visible again, can be further detailed and specified in an automated manner by area checks around the section 91 in question (potential scotoman area) by applying, for example, the following process steps:

An area 92 is defined around the sections 91 (potential scotoma or defect areas) detected during the first measurement run (cf. FIG. 8), through which the test spot is guided along a further path 70, again detecting the current positions 9 at which the test spot 6 becomes invisible or becomes visible again (cf. FIG. 9). After a further measurement pass along the further path 70 in the area 92 in question, the sections 91 (new potential loss areas) located in each case at the edge of the area 92 are used as the center for further areas 92 and these areas 92 are passed through again until there is an overlap with a further section 91 (potential scotoman area) detected in the first measurement pass or in the further measurement passes. This section 91 is also selected as the center of an area 92 that is traveled through according to the same principle (see FIGS. 9 to 11).

As an alternative to this procedure, the sections 91 (or potential scotoma or defect areas) identified during the first measurement run (cf. FIG. 8) can each be used as the center of an area 92 in which the test spot 6 is guided along a further path 70 in each case, again storing the current positions 9 at which the test spot 6 becomes invisible or visible again to the person P being examined (see FIGS. 9 and 11).

In particular, the areas 92 have an area that is smaller than the area that the path 7 sweeps during the first measurement pass. In particular, the respective area 92 can cover an area around the respective center (section 91) that corresponds to a visual angle of 5°, in particular 2.5°, in particular 1°, with the distance between adjacent parallel sections 70a of the further path 70 being 2.5° in each case, in particular 1° in each case, in particular 0.5° in each case.

With regard to further measurement runs, patient data of further persons can also be used, e.g. to train an AI algorithm. In this way, potential loss areas or sections 91 can be identified that were not found in the previous examination methods. For this purpose, among other things, two basic AI methods can be applied, namely, on the one hand, statistical analysis based on the stored current positions 9 in the coordinate system of the examination area (e.g., (x,y) coordinates in the coordinate system of the area display or further area display), and, on the other hand, image analysis of the visualized examination results, i.e., e.g., of the area 90 whose edges are formed by the positions 9. Based on the results of the AI analysis of other persons and the probability of a potential defect/scotoma in comparison with other persons, further areas 92 can be automatically examined, where potentially further, not yet recognized sections 91 can be located. For this purpose, as described above, further measurement runs can be performed in areas 92 determined by the AI (cf. FIG. 12).

Furthermore, the respective area 92 can also be determined by the examiner, after which an (automatic) further measurement run can be performed along a further path 70 in the manner described above (see FIG. 13).

Finally, it is also possible for the examiner to determine or control the further path 70 for the further measurement run in real time (see FIG. 14).

In all the above-mentioned examination methods or further measurement runs, the examiner can optionally influence or adjust the following examination parameters, i.e. in particular:
A size or diameter of the test spot 6 (e.g., start and end sizes of the test spot 6 and, if applicable, a change in size or rate thereof),
A speed of the moving test spot 6,
A direction of the path 7, 70 to be traveled through (e.g. horizontal, vertical, diagonal etc.)
A number of sections 70a of a path 7, 70 to be traveled through in an area 92 or on the area display or the further area display.

1. A method for measuring the visual field of a person (P) by means of a device (1), comprising the steps:
displaying a visually detectable test spot (6) on an area display (2) of the device (1) viewed by the person (P) by means of an eye of the person (P), wherein a spatial position of the head of the person (P) with respect to the area display (2) remains unchanged, and
Moving the test spot (6) on the area display (2) by means of the device (1) along a path (7) during a measurement run and triggering an interaction device (8) of the device (1) by the person (P) when the displayed test spot (6) becomes invisible to the person (P) or becomes visible again during its movement along the path (7) at a current position (9), wherein the device (1) is caused by the triggering of the interaction device (8) to store the respective current position as well as associated information from which it can be derived whether the test spot (6) has become invisible to the person (P) at the current position or has become visible again.

2. The method of claim 1, wherein the method comprises the further step of:
Displaying information and/or an area (90) of the person's (P) field of view on the area display (2) and/or on a further area display (3), said current positions forming edge points of the area (90).

3. The method according to claim 1 or 2, wherein the test spot (6) has a speed S/f in cm/s when moving on the display (2), wherein S is the distance traveled by the test spot in cm, and wherein f is a number in the range from 2 to 7, in particular in the range from 3 to 6, in particular in the range from 4 to 5, wherein f is in particular 4.7.

4. The method according to one of claims 1 to 3, wherein the eye of the person (P), when moving the test spot (6) along the path (7), has a distance (A) to the area display (2) which is in a range from 10 cm to 400 cm, in particular in a range from 20 cm to 200 cm, in particular in a range from 30 cm to 100 cm, in particular in a range from 30 cm to 50 cm, in particular in a range from 35 cm to 45 cm, wherein the distance is in particular 40 cm.

5. The method according to one of the preceding claims, wherein during the measurement run within a period of 1 minute at least 500 to 50000 different locations in the field of view are tested by means of the test spot (6), in particular at least 1000 to 25000 different locations, in particular at least 1500 to 10000 different locations, in particular at least 2500 different locations, and/or wherein the path (7) has a total length of at least 17.625 cm to 705 cm, and/or wherein the path (7) has a total length of at least 35.25 cm to 352.5 cm, and/or wherein the path (7) has a total length of at least 52.875 cm to 176.25 cm, and/or wherein the path (7) has a total length of at least 70.5 cm, wherein the test spot (6) travels along the entire path (7) during the measurement process within a period of time which is less than or equal to 1 minute.

6. The method according to one of the preceding claims, wherein the path (7) comprises a plurality of mutually parallel first sections (70), and/or wherein the path (7) comprises a plurality of mutually parallel second sections (71).

7. The method according to claim 6, wherein the first portions (70) intersect the second portions (71) such that, in particular, the first and second portions define a grid.

8. The method according to one of the claims 6 to 7, wherein the test patch (6) is first moved along each of the first sections (70) and then moved along each of the second sections (71).

9. The method according to one of the claims 6 to 8, wherein the first sections (70) extend vertically on the area display (2), and wherein the second sections (71) extend horizontally on the area display (2); or wherein the first sections extend horizontally on the area display, and wherein the second sections extend vertically on the area display.

10. The method according to one of the preceding claims, characterized in that the path (7) has at least one section (72) running along the vertical or inclined to the vertical; and/or in that the path (7) has at least one section running in an arcuate, in particular semicircular, shape; and/or in that the path (7) has at least one section (72) crossing nerve fibers (N) and running in particular orthogonally to the nerve fibers (N).

11. The method according to claim 2 or according to one of the claims 3 to 11 insofar referring to claim 2, wherein for visualization of the area (90) the current positions (9) at which the test spot (6) has become invisible are connected to a line which is displayed as a border line of the area (90) on the further area display (3), and/or in that the current positions (9) at which the test spot (6) has become visible are connected to a line which is displayed as a border line of the area (90) on the further area display (3), wherein in particular the area (90) surrounded by the border lines is displayed on the further area display (3) optically differentiable from the background of the further area display (3).

12. The method according to claim 2 or according to one of claims 3 to 11 insofar referring to claim 2, wherein the detected area (90) is checked by guiding the test sport (6) on the area display (2) under control of an examiner (U) and under observation by the person repeatedly out of the detected area (90) into a surrounding area in which the test spot (6) is visible to the person.

13. The method according to one of the preceding claims, wherein during the measurement run a central object (10) is displayed on the area display (2), in particular in the form of a cross, which is weaker in light than the test spot (6), and which is viewed by the person (P) to fix a viewing direction of the person (P), wherein the device (1) detects during the measurement run whether a viewing direction of the person (P) deviates from the central object (10) and stops the movement of the test spot (6) if a deviation is detected, wherein in particular the test spot (6) has a diameter which becomes smaller with decreasing distance from the central object (10).

14. The method according to claim 3 or according to one of claims 4 to 13 insofar referring to claim 3, wherein the speed of the test spot (6) is temporarily slowed down as required by the examiner, in particular by interaction with a user interface of the device (1), in particular to specify a current position at which the test spot (6) has become invisible or has become visible again.

15. The method according to one of the preceding claims, wherein the method further comprises the step of: performing a further measurement run for each section (91) of the path (7) of the measurement run extending between two adjacent stored current positions (9), in which in each case the test spot (6) on the area display (2) is moved by means of the device (1) along a further path (70) within an area (92) on the area display which contains the respective section (91) as a center, and in each case triggering of the interaction device (8) of the device (1) by the person (P), when the displayed test spot (6) becomes invisible to the person (P) during its movement along the further path (70) within the area (92) at a current position (9) or becomes visible again, wherein the device (1) is caused by the triggering of the interaction device (8) to store the respective current position (9) of the test spot (6) in the area (92) as well as associated information from which it can be derived whether the test spot (6) at the current position (9) has become invisible to the person (P) or visible again.

16. Method according to claim 15, wherein after the respective further measurement run for sections (91) of the further path (70) found in the respective area, which in each case extend between two adjacent stored current positions (9) of the further path (70) and lie at an edge of the area (92), further measurement runs are carried out until in the process a section (91) is detected which was previously found during the measurement run or during a further measurement run, wherein a further measurement run is also carried out for this section (91).

17. Method according to one of the claims 1 to 14, wherein at least one area (92) is selected automatically by means of an AI algorithm which has been trained with a multiplicity of data records of different persons, wherein a further measurement run is carried out in which in each case the test spot (6) on the area display (2) is moved by means of the device (1) along a further path (70) within the area (92) on the area display, wherein in each case the interaction device (8) of the device (1) is triggered by the person (P), when the displayed test spot (6), during its movement along the further path (70) within the area, becomes invisible to the person (P) at a current position (9) or becomes visible again, wherein the device (1) is caused by the triggering of the interaction device (8) to store the respective current position (9) of the test spot (6) in the area (92) as well as associated information from which it can be derived whether the test spot (6) at the current position has become invisible to the person (P) or visible again.

18. The method of claim 17, wherein the AI algorithm is configured to select the at least one area (92) based on records of different people, wherein the respective record of a person comprises the stored current positions (9) of the person, and/or wherein the AI algorithm is configured to select the at least one area (92) based on records of different people, wherein the respective record corresponds to the visualized area (90) of a person.

19. The method according to one of the preceding claims, the method further comprising the step of: performing at least one further measurement run for an area selected by the examiner, wherein the test spot (6) on the area display (2) is moved along a further path (7) within an area on the area display by means of the device (1), and in each case triggering of the interaction device (8) of the device (1) by the person (P), when the displayed test spot (6), during its movement along the further path (7) within the area, becomes invisible to the person (P) at a current position (9) or becomes visible again, wherein the device (1) is caused by the triggering of the interaction device (8) to store the respective current position of the test spot in the area as well as associated information from which it can be derived whether the test spot (6) at the current position has become invisible to the person (P) or visible again.

20. The method according to one of the preceding claims, the method further comprising the step of: Performing at least one further measurement run, wherein the test spot (6) on the area display (2) is moved along a further path (70) under the control of the examiner by means of the device (1), and in each case triggering of the interaction device (8) of the device (1) by the person (P) when the displayed test spot (6) becomes invisible to the person (P) during its movement along the further path (70) within the area at a current position (9) or becomes visible again, wherein the device (1) is caused by the triggering of the interaction device (8) to store the respective current position of the test spot in the area as well as associated information from which it can be derived whether the test spot (6) has become invisible to the person (P) at the current position or has become visible again.

21. The method according to claim 2 or according to one of claims 3 to 20 insofar referring to claim 2, wherein a content of the area display (2) is transmitted to the further area display (3) via a data transmission link (V) and/or wherein a content of the further area display (3) is transmitted to the area display (2) via a data transmission link (V).

22. The method according to one of the preceding claims, wherein the device (1) comprises a processing unit (5, 55), in particular for displaying and moving the visually detectable test spot (6) on the area display (2).

23. The method according to claim 22, wherein the processing unit (5) is a local processing unit at the location of the person (P), and wherein the respective current position as well as the associated information are stored on the local processing unit (5) and are transmitted to a further processing unit (55) via a data transmission link (V) and are evaluated on the further processing unit (55) to generate a result data set.

24. The method according to claim 22, wherein the processing unit is a local processing unit at the location of the person (P), and wherein the respective current position as well as the associated information are stored on the local processing unit (5) and are evaluated on the local processing unit (5) to generate a result data set, wherein the result data set is optionally transmitted to a further processing unit (55) via a data transmission link (V).

25. The method according to claim 22, wherein the device (1) further comprises a local processing unit (5) at the location of the person (P), which is connected to the area display (2), wherein the processing unit (55) causes the display and movement of the visually detectable test spot on the area display (2) via a data transmission link (V) to the local processing unit (5), wherein the respective current position as well as the associated information are stored on the processing unit (55) and evaluated to generate a result data set.

26. A computer program comprising instructions that, when the computer program is executed on the processing unit, cause the processing unit to perform the steps of the method according to one of the claims 22 to 25.

27. A device (1) for measuring the visual field of a person (P), comprising:
an area display (2) configured to be viewed by the person (P),
a processing unit (5, 55) configured to display a test spot (6) on the area display (2) to the person (P) and to move it along a predefinable path (7) on the area display (2), an interaction device (8) configured to be triggered by the person (P) when the displayed test spot (6) becomes invisible to the person (P) during its movement along the path (7) at a current position or becomes visible again, wherein the processing unit (5) is configured to store the respective current position and associated information when the interaction device (8) is triggered, from which information it is derivable whether the test spot (6) has become invisible to the person (P) at the current position or has become visible again.

28. The device according to claim 27, wherein the device (1) comprises a further area display (3) configured to be viewed by an examiner (U),

29. The device according to claim 28, wherein the processing unit (5) is further configured to display an area (90) of the person's (P) field of view on the further area display (3), said current positions (9) forming edge points of the area (90).

30. The device according to one of claims 27 to 29, wherein the device (1) comprises a fixation unit (4) configured to fix a head position of the person (P) with respect to the area display (2).

31. The device according to one of claims 27 to 30, wherein the device (1) comprises a further processing unit (55), and wherein the processing unit (5) is configured to transmit the current positions as well as the associated information to the further processing unit (55) via a data transmission link (V), wherein the further processing unit (55) is configured to evaluate the current positions as well as the associated information to generate a result data set.

32. The device according to one of claims 27 to 30, wherein the device (1) comprises a further processing unit (55), and wherein the processing unit (5) is configured to evaluate the current positions and the associated information to generate a result data set and to transmit the result data set to the further processing unit (55) via a data transmission link (V).

33. The device according to one of claims 27 to 30, wherein the device further comprises a local processing unit (5) at the location of the person (P), which is connected to the area display (2), wherein the processing unit (55) causes the display and movement of the visually detectable test spot on the area display (2) via a data transmission link (V) to the local processing unit (5), wherein the respective current position as well as the associated information are stored on the processing unit (55) and evaluated to generate a result data set.
